(19) United States
(12) Patent Application Publication
(10) Pub. No.: US 2006/0205818 A1
(43) Pub. Date: Sep. 14, 2006

(54) Method for the treatment of von Hippel-Lindau (VHL) disease with phenylacetyl-derivatives

(76) Inventor: Stanislaw R. Burzynski, Houston, TX (US)

Correspondence Address: HOWREY LLP, C/O IP DOCKETING DEPARTMENT, 2941 FAIRVIEW PARK DRIVE, SUITE 200, FALLS CHURCH, VA, 22042-7195, US

(21) Appl. No.: 11/365,289

(22) Filed: Mar. 1, 2006

Related U.S. Application Data
(60) Provisional application No. 60/659,759, filed on Mar. 8, 2005.

(51) Int. Cl. A61K 31/198; A61K 31/192

(52) U.S. Cl. 514/563; 514/570

(57) ABSTRACT

Provided are methods of treating von Hippel-Lindau disease (VHL). Specifically embodiments of the invention provide methods for the treatment of a patient afflicted with VHL using phenylacetyl-derivatives. Preferred embodiments of the invention provide for the use of phenylacetic acid (or its sodium salt), phenylacetylglutamine (or its sodium salt) and/or phenylacetylisoglutamine (or its sodium salt) to treat VHL. Other embodiments of the invention provide for the use of phenylacetyl-derivatives for the manufacture of a medicament for the treatment of VHL.

This application claims benefit of priority to provisional application Ser. No. 60/659,759 filed Mar. 8, 2005, the entire contents of which are incorporated by reference herein.

### FIELD OF THE INVENTION

The invention relates to methods for treating von Hippel-Lindau disease (VHL) and, more specifically, to treating VHL using phenylacetyl-derivatives.

### BACKGROUND OF THE INVENTION

VHL (also known as Cerebroretinal angiomatosis) is comparatively rare genetic disorder characterized by the abnormal growth of tumors in certain parts of the body. The tumors of the central nervous system (CNS) are typically low grade and are comprised of a nest of abnormal blood vessels (called hemangioblastomas, or angiomas in the eye); pheochromocytoma; and endolymphatic sac tumors. Hemangioblastomas may develop in the brain, the retina of the eyes, and other areas of the nervous system and can cause death, when located in the brain stem. Other types of tumors develop in the adrenal glands, the kidneys (renal cell carcinoma occurs in about 40% of individuals with VHL), the pancreas, or other viscera. Symptoms of VHL vary among patients and depend on the size and location of the tumors. Symptoms may include headaches, problems with balance and walking (ataxia), dizziness, weakness of the limbs, vision problems, high blood pressure, seizures, and mental retardation. Cysts (fluid-filled sacs) and/or tumors (benign or cancerous) may develop around the hemangioblastomas and cause the symptoms listed above (Filling-Katz et al., 1991). Pheochromocytomas may be asymptomatic or may cause sustained or episodic hypertension. Endolymphatic sac tumors can cause hearing loss of varying severity, which is often a presenting symptom (Choo et al. 1994).

VHL is inherited in an autosomal dominant manner and testing of the von Hippel-Lindau tumor suppressor gene (VHL gene) detects mutations in virtually 100% of the individuals diagnosed with VHL. Molecular genetic analysis indicates that point mutations account for approximately 72% of VHL mutations, with partial or complete gene deletion accounting for the remaining 28% (Stolle et al.). It is estimated that 80% of individuals with VHL inherited it from an affected parent; while the remaining 20% have the disease as the result of a de novo gene mutation. The offspring of an affected individual have 50% risk of inheriting the disease. While the prevalence of VHL is not certain, recent data suggest that there may be thousands of people afflicted with this malady.

Additionally, the VHL gene (which resides on chromosome 3p25) is mutated or silenced in >50% of sporadic renal cell carcinomas (Kaelin, 2004) and there is evince that VHL gene mutation is a step in the progression to pancreatic islet cell tumors (Lott, et al., 2002)

Current used methods for treating VHL vary depending on the size and location of the tumor. They include standard surgical removal, gamma knife surgery, diathermy, xenon, laser, and cryocoagulation (Raja et al., 2004; Shingleton & Sewell, 2002). However, due to complexities including variations in the location and size of VHL-associated tumors, these modes of treatment are frequently impractical or ineffective.

Other treatments for VHL are currently being investigated and include the uses of vascular endothelial cell growth factor inhibitors (George and Kaelin, 2003; Aiello et al. 2002), other antiangiogenic drugs (e.g. Madhusudan, et al., 2004), and hypo-methylating agents, such as zebularine and 5-aza-2′-deoxycytidine (Alleman et al., 2004). However, none of these treatments have yet proven effective (for a more detailed summary of VHL, its believed causes, treatments, and prognosis see the VHL web site at www.geneclinics.org/profiles/vhl). Thus, there exists a need for an effective method of treating VHL and its symptoms.

### SUMMARY OF THE INVENTION

Various embodiments of the invention provide for methods of treating patients believed to be suffering from von Hippel-Lindau disease (VHL) using an effective amount of pharmaceutical compositions comprising phenylacetylglutamine (or a pharmaceutically acceptable salt thereof), phenylacetylisoglutamine (or a pharmaceutically acceptable salt thereof), and/or phenylacetate (or a pharmaceutically acceptable salt thereof).

Other embodiments of the invention provide for the use of a composition comprising phenylacetylglutamine (or a pharmaceutically acceptable salt thereof), phenylacetylisoglutamine (or a pharmaceutically acceptable salt thereof), and/or phenylacetate (or a pharmaceutically acceptable salt thereof) for the manufacture of a medicament for treating a patient suspected of being afflicted with VHL.

### DEFINITIONS

The following definitions are provided in order to aid those skilled in the art in understanding the detailed description of the present invention.

As used herein the term “phenylacetyl-derivative” preferably refers to composition comprising phenylacetate and/or one or more analogs thereof that have been demonstrated to possess anti-cancer properties.

As used herein the term “pharmaceutically acceptable salt” means salts having the biological activity of the parent compound and lacking toxic activity at the selected administration level. Determination of whether a salt is pharmaceutically acceptable can be accomplished by methods known to those of skill in the art. By way of example only, pharmaceutically acceptable salts of phenylacetylglutamine, phenylacetylisoglutamine, and phenylacetic acid include, but are not limited to, inorganic sodium, potassium and ammonium salts, and organic diethanolamine, cyclohexylamine, and amino acid salts.

### ILLUSTRATIVE EMBODIMENTS OF THE INVENTION

Various embodiments of the instant invention provide methods of treating a patient, preferably a human patient, believed to be afflicted with von Hippel-Lindau disease (VHL) (also known as von Hippel-Lindau syndrome). More specifically, these embodiments of the invention provide methods of using phenylacetyl-derivatives to treat a patient believed to be afflicted with VHL. In preferred aspects of these embodiments, the patient(s) is/are treated with a combination of (a) phenylacetylglutamine (or a pharmaceutically acceptable salt thereof), (b) phenylacetylisoglutamine (or a pharmaceutically acceptable salt thereof) and (c) phenylacetic acid (or a pharmaceutically acceptable salt thereof). Hereafter, whenever the terms phenylacetylglutamine (PG), phenylacetylisoglutamine (isoPG), and phenylacetic acid (PN) are used, they should be viewed as encompassing both the acidic and pharmaceutically acceptable salt forms of the molecules.

Phenylacetylglutamine has a molecular weight of 264.28, its empirical formula is C₁₆H₁₆N₂O₄, and the structural formula provided below as Formula I.

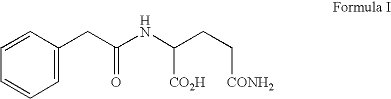

Phenylacetylisoglutamine has a molecular weight of 264.28; its empirical formula is C₁₆H₁₆N₂O₄, and the structural formula provided below as Formula II.

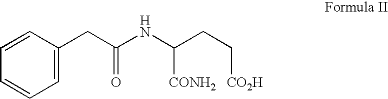

Phenylacetic acid has a molecular weight of 136.14, its empirical formula is C₈H₈O₂, and the structural formula is provided below as Formula III.

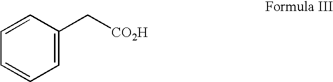

Various embodiments of the invention provide methods for treating a patient diagnosed with VHL using combinations of PG, isoPG, and/or PN. In certain aspects of these embodiments the PG, isoPG, and/or PN may be present in their acidic forms or as pharmaceutically acceptable salts.

In preferred aspects of these embodiments the patient is treated with a composition comprising a combination of PG, isoPG, and PN. Preferably, the composition comprises the sodium salts of PG, isoPG, and PN.

A more preferred embodiment of the invention comprises administering two pharmaceutical compositions to the patient. According various aspects of this embodiment, the first pharmaceutical composition comprising a combination of PG and isoPG and the second pharmaceutical composition comprises PN and PG. Preferably each compound in the first and second pharmaceutical compositions is present in its sodium salt form. An even more preferred aspect of this embodiment provides that the first pharmaceutical composition comprises PG and isoPG present at about a 4:1 ratio and that the second pharmaceutical composition comprises PN and PG present at about a 4:1 ratio.

Various aspects of these embodiment provide for administering the first and second pharmaceutical composition by any appropriate and effective means known in the art, including, but not limited to: orally, intravenously or by some other parenteral means. In preferred aspects of this embodiment the first and second pharmaceutical composition are delivered orally and/or intravenously. In a particularly preferred aspect of this embodiment the first and second pharmaceutical composition are delivered intravenously as aqueous solutions. Various aspects of the embodiment provide for either sequential or simultaneous administration of the first and second pharmaceutical compositions.

In various aspects of this embodiment the first and second pharmaceutical compositions are delivered as aqueous solutions with the first pharmaceutical compositions comprising PG and isoPG present at about a 4:1 ratio and the second pharmaceutical composition comprising PN and PG present at a 4:1 ratio. In particularly preferred aspects of this embodiment the combined concentration of PG and isoPG in the first pharmaceutical is from about 50 to about 400 mg/ml; preferably from about 200 to about 350 mg/ml; even more preferably about 300 mg/ml. Similarly, preferred aspects of this embodiment provide for combined concentration of PN and PG in the second pharmaceutical composition of from about 10 to about 120 mg/ml; preferably, from about 40 to about 100 mg/ml; even more preferably about 80 mg/ml.

Various aspects of this embodiment of the invention provide for intravenous delivery of these first and second aqueous pharmaceutical compositions at infusion rates of from about 20 to about 400 ml/hour; preferably the infusion rates are from about 50 to about 250 ml/hour. Various aspects of this embodiment provide for infusion frequencies of from once every other day to 10 times, or more, daily. In a preferred aspect of this embodiment the infusions are given every 4 to 6 hours.

According to various aspects of this embodiment of the invention the first pharmaceutical composition (combination of PG and isoPG) is delivered at a dose of from about 0.5 to about 25 g/kg/day; preferably from about 5 to about 20 g/kg/day. The second pharmaceutical composition is delivered at a dose of from about 0.02 to about 1 g/kg/day; preferably from about 0.1 to about 0.4 g/kg/day.

In a particularly preferred embodiment the patient diagnosed with VHL is treated with a combination of two pharmaceutical compositions. The first pharmaceutical composition comprises an aqueous solution of sodium phenylacetylglutaminate and sodium phenylacetylisoglutaminate in a 4:1 ratio and at a combined concentration of about 300 mg/ml. The second pharmaceutical composition comprises an aqueous solution of sodium phenylacetate and sodium phenylacetylglutaminate present in about a 4:1 ratio at a concentration of about 80 mg/ml. In various aspects of this embodiment of the invention the first and second pharmaceutical compositions are given sequentially every 4 to 6 hours to give a daily dosage of about 10-20 g/kg of the first pharmaceutical composition and about 0.2-0.4 g/kg of the second pharmaceutical composition. Various aspects of this invention provide for dosing occur at any effective frequency and for any effective/necessary duration. For example, daily dosing may continue for one week, two weeks, three weeks, one month, one year, or longer, as required. The decision as to the duration for treatment will depend on the individual patient's response and must would likely be make by the patient's physician after evaluation of the patient's response to the treatment.

Other embodiments of the invention provide for treatment of a patient diagnosed with VHL using an effective amount of PG, isoPG, and PN administered orally. In preferred aspects of this embodiment, the patient is given two pharmaceuticals, the first pharmaceutical comprising PG and isoPG in a 4:1 ratio and the second pharmaceutical comprising PN and PG in a 4:1 ratio. The first and second pharmaceutical compositions may be orally delivered in any suitable form including, but not limited to: capsules, tablets, as a bolus, as a liquid solution or liquid suspension or using any suitable combination of these delivery forms. In various aspects of this embodiment treatment of the patient using oral delivery may be made prior to, concomitant with, or subsequent to treating the patient using another delivery form (e.g. intravenous delivery). One preferred aspect of this embodiment provides for treating the patient with a first treatment regiment comprising delivering two pharmaceutical compositions intravenously, the first pharmaceutical composition comprising PG and isoPG and the second pharmaceutical composition comprising PN and PG. At a different time, either prior to or subsequent to the first treatment regimen, the patient is treated with a second treatment regimen. This second treatment regimen comprises orally administering third and fourth pharmaceutical compositions to the patient. The third pharmaceutical composition comprising PG and isoPG and the fourth pharmaceutical composition comprising PN and PG.

It is noted that in all aspects of the invention drawn to the use of PG, isoPG, and PN to treat VHL contemplate the use of the acidic form and/or a pharmaceutically acceptable salt form of each the compounds, with the sodium salt representing a preferred embodiment. Moreover, all possible combinations of the compounds, concentrations, delivery (e.g. infusion) rates, dosages, and treatment regimens described herein are part of the methods provided by the instant invention.

Other embodiments of the invention provide for the use of PG, isoPG, and/or PN for the manufacture of a pharmaceutical formulation effective for the treatment of von Hippel-Lindau disease. Various aspects of these embodiments provide for the use of the acidic form and/or a pharmaceutically acceptable salt for of each compound; if a salt form, the compound is preferably a sodium salt.

According to various aspects of this embodiment PG, isoPG, and PN are used to prepare two medicaments. The first medicament comprising PG and isoPG in a 4:1 ratio and the second medicament comprising PN and PG in a 4:1 ratio. In specific aspects of this embodiment, the medicament may be prepared for either oral (e.g. a bolus, capsule, tablet, suspension, or other suitable form) delivery or parenteral delivery (e.g. an sterile aqueous solution). In preferred aspects of this embodiment the first and second medicaments are aqueous solutions with the first medicament comprising PG and isoPG at a 4:1 ratio with a combined concentration of from 50-400 mg/ml; preferably 300 mg/ml. The second medicament comprises PN and PG in a 4:1 ratio with a combined concentration of from 10-120 mg/ml; preferably 80 mg/ml. In all aspects of this embodiment the medicaments are suitable for either simultaneous or sequential delivery at an infusion rate of from 20-400 ml/hour; preferably from 50-250 ml/hour.

In other embodiments of the invention the methods of treating patients with VHL may comprise using a pro-drug and/or a pro-drug may be used to prepare a medicament to treat VHL. Suitable pro-drugs include 3-phenylacetylamino-2,6-piperidinedione (PP) (Formula IV) and phenylbutyrate (salt of phenylbutyric acid) (PB) (Formula V). 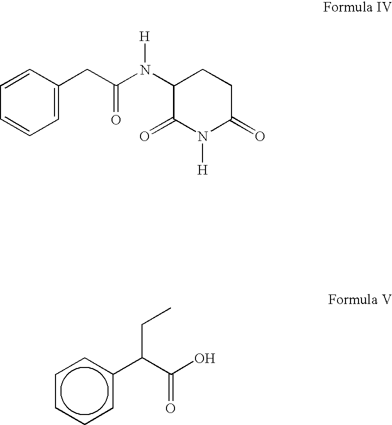
PP is metabolized in the small intestine to a combination of PG and isoPG. Similarly, PB is metabolized in the liver to a combination of PG and PN. Accordingly, various aspects of the instant inventions contemplate the use of pro-drugs that are metabolized to PG, isoPG, and/or PN, for treatment of patients believed to be suffering from VHL and for preparing such pro-drugs for the manufacture of a medicament effective for the treatment of VHL.

### EXAMPLES

The following examples are included to demonstrate preferred embodiments of the invention. It should be appreciated by those of skill in the art that the techniques disclosed in the examples which follow represent techniques discovered by the INVENTOR/S to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Treatment of 40-Year Old Caucasian VHL Patient with Phenylacetyl-Derivatives

The patient, a 40-year old Caucasian male, developed ataxia, aphasia, and right facial nerve paralysis in January 2003. MRI and PET scans revealed progressive lesions in the brain stem, cerebellum and right internal capsule described as hemangioblastomas and a CT scan has shown a left renal cyst. A brain biopsy was not recommended because of associated risk.

In July 2003, molecular genetic study (MGS) for von Hippel-Lindau disease was performed at the Children's Hospital in Philadelphia (CHP) using whole blood as the tissue sample. The laboratory results of this testing indicate that the testing was performed as follows. First genomic DNA was extracted from peripheral blood. Next, the genomic DNA was digested with Eco RI and Ase I restriction endonucleases and the digested DNA was subjected to Southern blot analysis with hybridization to probes specific for the VHL gene (this was done to detect complete or partial deletions, following the methods previously described by Stolle et al., 1998. In this analysis the normal VHL gene fragment presents as a 9.7 kilobase DNA fragment.

Additionally, exons of the VHL gene were amplified by polymerase chain reaction (PCR) using primers indicated in Stolle et al., 1998 and then subjected to conformation sensitive gel electrophoresis (Ganguly et al., 1993) to screen for the presence of point mutations. The DNA fragments were sequenced and their sequences compared with controls.

The July 2003 MGS report indicates that the Southern blot analysis for the patient is consistent with the complete deletion of one allele of the VHL gene.

On Jul. 30, 2003 the patient was admitted for intravenous administration of phenylacetyl-derivatives in accordance with Federal Drug Administration approved protocol BT-11. Pharmaceutical compositions comprising PG:isoPG (4:1) and PG:PN (1:4), respectively, were administered intravenously at a dosage of 10.72 g/kg/day and 0.33 g/kg/day respectively until Jan. 25, 2004. From May 9, 2004 to Jul. 13, 2004 the patient was given both compositions orally (0.15 g/kg/day). No corticosteroids were administered during the treatment with phenylacetyl-derivatives.

After 3 months of treatment with the phenylacetyl-derivatives the ataxia, aphasia, and facial paralysis were no longer evident. Moreover, a PET scan after 5 months of treatment and an MRI scan done at 11 and 15 months after the commencement of treatment, were within normal limits. Moreover, follow up MGS done at CHP after 3 months of treatment failed to identify a detectable deletion or point mutation in the patient's VHL gene.

### Example 2

### Preparation and Administration Phenylacetyl-Derivatives

Phenylacetyl-derivatives, specifically PG, isoPG, and PN, may be obtained by any means known to those of art skill in the art. For example see U.S. Pat. No. 6,258,849 (Burzynski), which is herein incorporated by reference. This patent describes the both the isolation of phenylacetyl-derivatives from human urine and the chemical synthesis of these derivatives.

For use in the treatment described in this example a phenylacetyl-derivative composition for injection as a sterile solution comprising sodium phenylacetylglutaminate (PG) and sodium phenylacetylisoglutaminate (isoPG) in an approximately 4:1 ratio, was prepared (PG/isoPG composition). The PG/isoPG composition was delivered intravenously at a concentration of ˜300 mg/ml (comprising ˜230-250 mg/ml of PG and ˜55-65 mg/ml of isoPG).

A PN/PG composition for injection is a sterile solution comprising sodium phenylacetate (PN) and PG in an approximately 4:1 ratio. The PN/PG composition was delivered intravenously at a concentration of ˜80 mg/ml (comprising ˜62-65 mg/ml of PN and ˜15-17 mg/ml of PG).

To prepare the PG/isoPG- and PN/PG-compositions for injection the PG, isoPG, and PN were dissolved in water and the pH was adjusted to ˜7.0 using sodium hydroxide.

Phenylacetyl-derivative compositions may be administered by any effective means including, but not limited to intravenously and orally. An exemplary dosage schedule for adults is as follows:

The individual injection may be given over a one-hour or longer period of time for a 70 kg patient, depending on the patient's tolerance. If a patient experiences a chemical taste in his/her mouth during the injection the flow rate of the pump should be decreased to 200 ml/hour. If the patient continues to experience the chemical taste, the flow rate should be decreased to 150 ml/h, if the taste persists the rate should be decreased in 50 ml/h increments to 100 ml/h or until the taste dissipates.

On the first day of administration of phenylacetyl-derivatives the intravenous (IV) pump may be loaded with 240 ml (˜72 g) of the PG/isoPG composition and 200 ml (˜16 g) of the PN/PG composition. The patient is first given 10 ml of the PG/isoPG composition at an infusion rate of 100 ml/h, followed in 15 minutes by 10 ml of the PN/PG composition at an infusion rate of 100 ml/h. Four hours from the beginning of the initial infusion, the patient is given 46 ml of the PG/isoPG composition at 250 ml/h followed by 38 ml of the PN/PG composition at 250 ml/h, the second infusion procedure is repeated every four hours.

Beginning with the second day the PG/isoPG-composition dose is increased daily in 20 ml increments until the highest tolerable or effective dose is reached, typically not exceeding 20 g/kg/day. Similarly, the PN/PG-composition dose is increased daily in 10 ml increments until the highest tolerable or effective dose is reached, typically not to exceed 0.4 g/kg/day.

### DOSAGE SCHEDULE FOR CHILDREN

The dosage schedule for children is different, depending on age. An exemplary dosage schedule is as follows.

On the first day of administration of phenylacetyl-derivatives, the flow rate of the pump is typically maintained at 25 ml/hour. Beginning with the second day of administration the individual injection will be given at 50 to 250 ml/hour, depending on the patient's age and tolerance. On the first day of administration the pump will be loaded with 60 ml of the PG/isoPG-composition and 60 ml of the PN/PG-composition. The volume of the injection will be approximately 10 ml every 4 hours (six times a day).
1. Six months to less than two years old: flow rate 50 ml/h
2. Two to less than four years old: flow rate 75 ml/h
3. Four to less than seven years old: flow rate 100 ml/h
4. Seven to less than 10 years old: flow rate 150 ml/h
5. 10 to less than 16 years old: flow rate 200 ml/h
6. 16 to 18 years old: flow rate 250 ml/h.

If the patient experiences a chemical taste in his/her mouth during the injection the flow rate will be decreased in 25 ml/h increments until the patient no longer experiences the chemical taste.

Beginning with the second day of treatment the phenylacetyl-derivatives may be administered as follows.
1. Less than 12 years old: the PG/isoPG-composition dose is increased daily, in 10 ml increments, until the highest tolerable or effective dose is reached, typically not exceeding 25 g/kg/day. The PN:PG-composition dose is increased daily, in 5 ml increments, until the highest tolerable or effective dose is reached, typically not exceeding 0.6 g/kg/day.
2. 12 years old to less than 16 years old: the PG/isoPG-composition dose is increased daily, in 20 ml increments, until the highest tolerable or effective dose is reached, typically not exceeding 25 g/kg/day. The PN/PG-composition dose is increased daily, in 5-10 ml increments, until the highest tolerable or effective dose is reached, typically not exceeding 0.6 g/kg/day.
3. 16 years old to less than 18 years old: PG/isoPG-composition dose is increased daily, in 20 ml increments, until the highest tolerable or effective dose is reached, typically not exceeding 25 g/kg/day. The PN/PG-composition dose is increased daily, in 10 ml increments, until the highest tolerable or effective dose is reached, typically not exceeding 0.6 g/kg/day.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods described herein without departing from the enabled concept of the invention. More specifically, it will be apparent that certain compounds that are both chemically and physiologically related may be substituted for the agents described herein while the same or similar results would be achieved. All such similar substitutes and modifications apparent to those skilled in the art are deemed to be within the scope of the invention.

### REFERENCES

The following references, to the extent that they provide exemplary procedural or other details supplementary to those set forth herein, are specifically incorporated herein by reference.
Aiello L P, George D J, Cahill M T, Wong J S, Cavallerano J, Hannah A L, Kaelin W G Jr (2002) Rapid and durable recovery of visual function in a patient with von hippel-lindau syndrome after systemic therapy with vascular endothelial growth factor receptor inhibitor su5416. Ophthalmology 109:1745-51.
Alleman W, Tabios R, Chandramouli G, Aprelikova O, Torres-Cabala C, Mendoza A, Rodgers C, Sopko N, Linehan W, Vasselli J (2004) The in vitro and in vivo effects of re-expressing methylated von Hippel-Lindau tumor suppressor gene in clear cell renal carcinoma with 5-Aza-2′-deoxycytidine. Clinical Cancer Res. 10:7011-7021.
Choo D, Shotland L, Mastroianni M, Glenn G, van Waes C, Linehan W M, Oldfield E H (2004) Endolymphatic sac tumors in von Hippel-Lindau disease. J Neurosurg 100:480-7
Filling-Katz M R, Choyke P L, Oldfield E, Chamas L, Patronas N J, Glenn G M,. Gorin M B, Morgan J K, Linehan W M, Seizinger B R, et al (1991) Central nervous system involvement in Von Hippel-Lindau disease. Neurology 41:41-6
Ganguly et al. (1993) Proc. Natl. Acad. Sci. USA 90:10325-10329.
George G and Kaelin W (2003) The von Hippel-Lindau protein, vascular endothelial growth factor, and kidney cancer. New England J. Medicine 349:419-421.
Kaelin, W. G. (2004) The Von-Hippel-Lindau tumor suppressor gene and kidney cancer. Clinical Cancer Research 10:6290s-6295s (supplement).
Lott S, Chandler D, Curley S, Foster C, El-Naggar A, Frazier M, Strong L, Lovell M, Killary A (2002) High frequency loss of heterozygosity in von Hippel-Lindau (VHL)-associated and sporadic pancreatic islet cell tumors: evidence for a stepwise mechanism for malignant conversion in VHL tumorigenesis. Cancer Research 62:1952-1955.
Madhusudan S, Deplanque G, Braybrooke J P, Cattell E, Taylor M, Price P, Tsaloumas M D, Moore N, Huson S M, Adams C, Frith P, Scigalla P, Harris A L (2004) Antiangiogenic therapy for von Hippel-Lindau disease. JAMA 291:943-4.
Raja D, Benz M S, Murray T G, Escalona-Benz E M, Markoe A (2004) Salvage external beam radiotherapy of retinal capillary hemangiomas secondary to von Hippel-Lindau disease: visual and anatomic outcomes. Ophthalmology 111:150-3.
Shingleton W B and Sewell P E Jr (2002) Percutaneous renal cryoablation of renal tumors in patients with von Hippel-Lindau disease. J Urol 167:1268-70.
Stolle C, Glenn G, Zbar B, Humphrey J S, Choyke P, Walther M, Pack S, Hurley K, Andrey C, Klausner R, Linehan W M (1998) Improved detection of germline mutations in the von Hippel-Lindau disease tumor suppressor gene. Hum Mutat 12:417-23.

What is claimed is:

1. A method for treating a patient diagnosed with von Hippel-Lindau disease, the method comprising administering to the patient an effective amount of phenylacetylglutamine (PG), or a salt of PG; phenylacetylisoglutamine (isoPG), or a salt of isoPG; and phenylacetic acid (PN), or a salt of PN.

2. The method of claim 1 comprising administering to the patient an effective amount of sodium phenylacetylglutaminate, sodium phenylacetylisoglutaminate, and sodium phenylacetate.

3. The method of claim 1 comprising administering to the patient:
a) a first pharmaceutical composition comprising phenylacetylglutamine (PG), or a salt of PG, and phenylacetylisoglutamine (isoPG), or a salt of isoPG, and
b) a second pharmaceutical composition comprising phenylacetic acid (PN), or a salt of PN, and PG, or a salt of PG.

4. The method of claim 3 wherein the first pharmaceutical composition comprises sodium phenylacetylglutaminate and sodium phenylacetylisoglutaminate in an about a 4:1 ration and the second pharmaceutical composition comprises sodium phenylacetate and sodium phenylacetylglutaminate in about a 4:1 ratio.

5. The method of claim 4 comprising administering the first and second pharmaceutical compositions orally.

6. The method of claim 4 comprising administering the first and second pharmaceutical compositions parenterally.

7. The method of claim 6 wherein the first and second pharmaceutical compositions are administered as solutions and wherein the combined concentration of sodium phenylacetylglutaminate and sodium phenylacetylisoglutaminate in the first pharmaceutical composition is between about 50 and about 400 mg/ml and the combined concentration of sodium phenylacetate and sodium phenylacetylglutaminate in the second pharmaceutical composition is between about 10 and about 120 mg/ml.

8. The method of claim 7 wherein the combined concentration of sodium phenylacetylglutaminate and sodium phenylacetylisoglutaminate in the first pharmaceutical composition is about 300 mg/ml and the combined concentration of sodium phenylacetate and sodium phenylacetylglutaminate in the second pharmaceutical composition is about 80 mg/ml.

9. The method of claim 8 comprising administering the first and second pharmaceutical compositions intravenously each at an infusion rate of from 20 ml/hr to 400 ml/hr.

10. The method of claim 9 wherein the infusion rate of the first and second pharmaceutical compositions is from about 50 ml/hour to about 250 ml/hour.

11. The method of claim 8 wherein the first and second pharmaceutical compositions are administered sequentially.

12. A method of treating a human patient diagnosed with von Hippel-Lindau disease, the method comprising:
administering intravenously a first pharmaceutical composition and a second pharmaceutical composition;
(a) wherein the first pharmaceutical composition comprises an aqueous solution of sodium phenylacetylglutaminate and sodium phenylacetylisoglutaminate present at a about 4:1 ratio and having a combined concentration of from about 50 to about 400 mg/ml;
(b) wherein the second pharmaceutical composition comprises an aqueous solution of sodium phenylacetate and sodium phenylacetylglutaminate present at a about 4:1 ratio and having a combined concentration of from about 20 to about 120 mg/ml;
wherein the first and second pharmaceutical are administered at an infusion rate of from about 20 to about 400 ml/hour.

13. The method of claim 12 wherein the concentration of the first pharmaceutical composition is about 300 mg/ml; the concentration of the second pharmaceutical composition is about 80 mg/ml and the infusion rate for the first and second pharmaceutical compositions is about 250 ml/hour

14. The method of claim 12 wherein the dose of the first pharmaceutical composition is from about 0.5 to about 25 g/kg/day and the dose of the second pharmaceutical composition is from about 0.02 g/kg/day to about 1 g/kg/day.

15. The method of claim 13 wherein the dose of the first pharmaceutical composition is from about 5 to about 20 g/kg/day and the dose of the second pharmaceutical composition is from about 0.1 to about 0.4 g/kg/day.

16. The method of claim 12 further comprising orally administering a third pharmaceutical composition and a fourth pharmaceutical composition to the patient; wherein the third composition comprises wherein the third pharmaceutical composition sodium phenylacetylglutaminate and sodium phenylacetylisoglutaminate present at a about 4:1 ratio and the fourth pharmaceutical composition comprises an sodium phenylacetate and sodium phenylacetylglutaminate present at a about 4:1 ratio.
